(19) United States
(12) Patent Application Publication
(10) Pub. No.: US 2022/0162274 A1
(43) Pub. Date: May 26, 2022

(54) PRODUCTION AND USES OF ARTIFICAL HISTONE H1 FOR ANALYZING, DIAGNOSING, TREATING, AND/OR PREVENTING SENESCENCE

(71) Applicant: QUALUS RESEARCH SpA, Santiago (CL)

(72) Inventor: Felipe Veloso, Santiago (CL)

(21) Appl. No.: 17/430,241

(22) PCT Filed: Feb. 11, 2020
(86) PCT No.: PCT/IB2020/051098

§ 371 Date: Aug. 11, 2021

Related U.S. Application Data
(60) Provisional application No. 62/803,987, filed on Feb. 11, 2019.

(51) Int. Cl. C07K 14/47; G16B 20/30; G16B 20/50; C07K 14/435

(52) U.S. Cl. CPC C07K 14/47; G16B 20/30; A61K 38/00; C07K 14/43545; G16B 20/50

(57) ABSTRACT

The present invention provides a method for producing artificial protein sequences and artificial nucleic acid sequences for the linker histone variants H1.0 (also known as histone H1°; H1(0); H5; H1δ; RI H1; or H1 histone family, member 0) and H1x (also known as histone H1.10 or H1 histone family, member X). In particular, the artificial protein sequences produced by the method feature engineered α-helical motifs—three structural motifs in the histone H1 that bind to nucleosomal and/or linker DNA in chromatin. These artificial-sequence histone H1 proteins, when they replace or supplement their wild-type counterparts in vivo, confer multicellular individuals significant resistance to senescence and/or age-related health conditions such as age-related cancer.

### PRODUCED AND USES THEREOF

This application claims the benefit of Provisional U.S. Patent Application No. 62/803,987, filed on Feb. 11, 2019. This provisional application is incorporated by reference herein in its entirety.

### TECHNICAL FIELD OF THE INVENTION

The present invention pertains to protein engineering. In particular, to a method for enhancing the DNA binding affinity of DNA-proximal regions within the globular motif known as “winged” helix-turn-helix (wHTH)—as α₁-β₁-α₂-α₃-β₂-β₃, where the αᵢ are alpha helices and the βⱼ are beta sheets—in the histone H1 protein (also known as linker histone) which in turn is critical for necessary higher-order constraints on chromatin dynamics. In the context of the major wHTH motif, the invention relates in particular to a method for enhancing the DNA-binding affinity of the histone H1 α-helical structural motifs α₃ (most preferred, which binds to both nucleosomal and linker DNA), α₂ (second most preferred, which binds to nucleosomal DNA), and α₁ (third most preferred, which binds to linker DNA, see FIG. 1).

The potential of this invention to treat and/or prevent senescence, cancer, and/or age-related health conditions in multicellular species encompasses clinical, cosmetic, industrial, and agricultural applications.

### BACKGROUND OF THE INVENTION

For centuries, intellectual endeavors have entertained the prospect of unlimited lifespan. Today, from services of cryonic suspension and storage of humans and pets to “anti-aging” skin care cosmetics, a variety of industries try to satisfy the increasing human demand to ameliorate or even overcome the accumulation of biological dysfunctions after adulthood that characterizes the senescence process. However, to humans and most other multicellular species, senescence remains an unstoppable process that inexorably leads to the death of the multicellular individual.

On the other hand, the group of diseases called cancer is a major cause of death worldwide, with 9.6 million estimated deaths in 2018. Also, over 70% of deaths from cancer take place in low- and middle-income countries. Additionally, the global economic burden of cancer in 2010 was estimated to be approximately US $1.16 trillion.

Current treatments for cancer include surgery, radiotherapy, chemotherapy, immunotherapy, targeted therapy, hormone therapy, stem cell transplant, and precision medicine. These treatments mainly aim to prolong the patient's life and in some cases to cure cancer occurrences (especially early detectable cancers). Nevertheless, a comprehensive and effective treatment for cancer, let alone its effective prevention, is still lacking.

Importantly for the present invention, there is a well-known strong positive correlation between cancer incidence and age after adulthood. With respect to said correlation, age-related cancer has been described as the result of a poorly tuned yet strong enough “pushback” of the multicellular individual against its own senescence process. As a consequence, it has been also suggested that stopping senescence and eliminating the incidence of age-related cancer are one and the same technical challenge.

In this context, what is needed is an invention that effectively confers the multicellular individual resistance (being this resistance prophylactic and/or therapeutic) to senescence and/or age-related health conditions, such as age-related cancer.

The specifics of the present invention will be presented in the next section.

With respect to the related state of the art, the patent application WO2005040814 addresses methods and means of cancer detection based on the determination of the presence or amount of post-translational modifications (PTMs) of residues within histone proteins, for example, methylation of lysine residues, in order to assess a cancer condition. Methylated lysine residues, which may be detected in these methods, include, for example, H3 Lys 27, H3 Lys 36, and H4 Lys 20. This document does not address H1 proteins or artificial sequences thereof in terms of their application in the treatment/prevention of senescence or cancer.

Additionally, document WO0151511 addresses the recombinant production of human histone H1 subtypes and their therapeutic uses for cancer, autoimmune diseases, endocrine disorders, and also their use as an antibiotic, where the protein or an active fragment of it is synthesized in E. coli. Importantly, this document never addresses any redesign or artificial sequence of the histone H1.0 or histone H1x protein α-helical motifs and its uses thereof.

WO0172784 shows therapeutic peptides having a motif that binds specifically to non-acetylated H3 and H4 histones for cancer therapy and compositions thereof. This invention is based on the anti-carcinogenic property of a chromatin binding peptide isolated from soybean seed having a highly conserved motif in other chromatin-binding proteins from different species, showing that it can be developed as an in vivo gene silencing technology for biological and medical research. Pharmaceutical compositions useful in retarding, stopping, or reducing various types of cancers are also described. The document never addresses the histone H1.0 or histone H1x proteins or artificial sequences thereof for the treatment/prevention of senescence, cancer, or age-related health conditions.

Finally, U.S. Pat. No. 8,962,562 claims a method for treating thrombocytopenia using at least one human recombinant histone, especially at least one histone H1 subtype. This document focuses on the histone H1.3 protein and in particular does not address the redesign of the histone H1.0 protein or the histone H1x protein nor the redesign of their respective α-helical motifs and uses thereof.

### SUMMARY OF THE INVENTION

The present invention is directed to a method for producing artificial protein and artificial nucleic acid sequences, where the artificial sequences are useful for the analysis, diagnosis, treatment, and/or prevention of senescence (also known as biological aging) and/or age-related health conditions, such as age-related cancer.

The artificial sequences produced through the method can be used on general research for better understanding of both the senescence and cancer phenomena in multicellular species.

The scope of the invention also encompasses any biomedical, cosmetic, industrial, and agricultural uses of the method according to the present invention.

This invention consists in a method that applies a set of amino acid substitutions, insertions and/or deletions to the wild-type histone H1.0 and histone H1x protein (or any respective protein ortholog) sequences wherein the set of amino acid substitutions, insertions and/or deletions entails an increase in the net electric charge (z) at physiological pH of the artificial-sequence protein DNA-binding regions (in particular, α-helices) with respect to their wild-type counterparts when these artificial-sequence and wild-type protein regions are each in their respective post-translationally unmodified forms or, particularly, when each region is respectively subjected to plausible post-translational modifications (PTMs), effectively creating a “reservoir” of positive electric charge in the artificial-sequence histone H1, which facilitates its electrostatic binding (especially when post-translationally modified in vivo) to the negatively charged DNA.

The present invention also encompasses the artificial sequences produced using the method and the uses thereof.

The method for producing artificial histone H1 sequences is useful for a number of biomedical, cosmetic, industrial, and/or agricultural purposes pertaining to senescence (also known as biological aging) and/or age-related health conditions, such as certain types of cancer.

Arginine is the most basic amino residue, has a high α-helical configuration propensity, and its positive electric charge (i.e., z>0) is virtually permanent at physiological pH.

In its post-translationally unmodified form, the lysine residue is positively charged (i.e., z>0) at physiological pH. Yet, when lysine undergoes post-translational acetylation it suffers a significant decrease in z. If lysine residues are present in histone H1 α-helical motifs then lysine post-translational acetylation is, in principle, possible. Thus, one or more arginine residues substituting a lysine residue known or predicted to undergo post-translational acetylation (or substituting residues proximal to said lysine residue) serve in the method as a “reservoir” of positive electric charge that stabilizes the electrostatic binding affinity of the artificial histone H1.0 and histone H1x proteins to the negatively charged nucleosomal and/or linker DNA.

The asparagine residue has relatively low α-helical configuration propensity yet it is still found in N- and C-terminal regions of α-helical motifs and, in particular, asparagine can be found at the N-terminus of the α₃-helix within H1.0 and H1x histones. Asparagine is electrically neutral at physiological pH but it may undergo a deamidation reaction, which converts it to the negatively charged aspartic acid. While infrequent, this deamidation reaction is significantly more likely when the asparagine residue is followed by a glycine residue.

Alanine and methionine are electrically neutral amino acid residues at physiological pH, display a high α-helical configuration propensity and, importantly, are not subject (given their properties and location in the histone H1 globular domain) to PTMs that significantly decrease their net electric charge (z). Thus, under this invention alanine and methionine are adequate substitute residues to be applied in the method to wild-type α-helical motifs.

The proline residue is electrically neutral at physiological pH and hydroxylation is the only PTM proline can be subjected to, which does not decrease its net electric charge (z). However, proline's unique properties make its helix-forming propensity extremely low. Thus, under this invention the proline residue is an adequate substitute only for residues located at the N-terminus of wild-type α-helical motifs (if located at the N-terminus of an α-helical motif, a proline residue is incapable of breaking/kinking said motif and may even stabilize it).

Post-translationally phosphorylated amino acid residues display a decreased electric charge at physiological pH with respect to their respective unmodified form because of the addition of the negatively charged phosphate group. Thus, under this invention, a phosphorylatable amino acid residue (i.e., serine, threonine, tyrosine, and histidine) within an α-helical motif may be deleted or, better yet, substituted with residues such as alanine, methionine, leucine, arginine, or proline (provided proline is substituting a phosphorylatable residue located at the N-terminus of the α-helix). Such an amino acid substitution in the method also creates a “reservoir” of positive electric charge with respect to the substituted wild-type amino acid residue when phosphorylated.

Wild-type α-helical motifs may display aspartic acid or glutamic acid residues, which are negatively charged (i.e., z<0) at physiological pH. Particularly when located nearby DNA-binding amino acid residues, a negatively charged residue can be deleted or, better yet, substituted with a residue such as alanine according to the present invention. A method substituting a negatively charged amino acid residue with one that is not effectively creates a “reservoir” of positive electric charge with respect to the net electric charge (z) of the wild-type α-helical motif at physiological pH.

Importantly, whereas the method entails the application of a set of amino acid substitutions, insertions, and/or deletions applied to wild-type histone H1.0 and histone H1x protein sequences, its preferred embodiment applies only amino acid substitutions (as opposed to amino acid insertions or deletions) at up to eleven specific sites. Alternatively, the method can also apply amino acid substitutions (other than the aforementioned), insertions, and/or deletions to the wild-type histone H1.0 or histone H1x protein sequences as long as the secondary and tertiary protein structures remain biologically functional.

At the multicellular-individual level, this invention is useful for providing significant resistance to senescence (also known as biological aging) and to age-related conditions (e.g., some types of cancer) by stabilizing the electrostatic binding affinity of the histone H1.0 and histone H1x to the negatively charged nucleosomal and/or linker DNA at the chromatin level, which in turn stabilizes the higher-order, histone H1-dependent constraints on chromatin dynamics. Said constraints are critical, when disrupted or dissipated, for the senescence and the age-related cancer phenomena.

It is also important to emphasize that, whereas the artificial histone H1.0 and histone H1x proteins produced by the method can be natively synthesized by genetically modified non-human species, for humans it is principally envisioned the extrinsic delivery of synthetic mRNA (encoding the artificial-sequence histone H1.0 and histone H1x proteins) to the cells. The future development of an effective “histone H1.0/H1x replacement/supplement therapy” (via gene suppression techniques and/or in vivo mRNA delivery) is plausible because (i) histone H1 proteins display a very high turnover rate in chromatin (the mean residence time of a histone H1 protein at its binding site has been estimated to be approximately 3 minutes) and (ii) both H1.0 and H1x histone variants are not tissue-specific, which should facilitate the delivery of the required synthetic mRNA in vivo.

### BRIEF DESCRIPTION OF THE DRAWING

For a fuller understanding of this invention, reference is made to the following description and accompanying drawing, in which:

FIG. 1 depicts the histone H1.0/H1x protein with its characteristic wHTH structural motif, the location and orientation of the α₁, α₂, and α₃ helical motifs (gray dashed lines) with respect to the nucleosomal and/or linker DNA they bind to, the location and orientation of the beta sheet motifs β₁, β₂, and β₃, and also the eleven amino acid substitution sites (S1, . . . , S11) used to produce artificial histone H1.0/H1x protein sequences according to the claimed method. Credits: FIG. 1 was created based on a publicly available 3D structure data file [PDB ID: 5NL0, I. Garcia-Saez, C. Petosa, S. Dimitrov (2017) Crystal structure of a 197-bp palindromic 601L nucleosome in complex with linker histone H1, doi: 10.2210/pdb5NL0/pdb].

### DETAILED DESCRIPTION OF THE INVENTION

In particular, the present invention provides a method for producing artificial protein and artificial nucleic acid sequences (such as RNA or DNA) for two histone H1 variants or any of their respective orthologs, wherein the method entails the application of a set of amino acid (aa) substitutions, insertions, and/or deletions to the respective wild-type (wt) protein sequence at clearly defined sites, and wherein the method entails the satisfaction of a very specific electrochemical condition for each artificial protein sequence produced by the method with respect to its wild-type counterpart.

In a preferred embodiment of the invention, the method entails the application of a set of amino acid substitutions, insertions, and/or deletions which in turn entails an increase in the net electric charge (z) (at physiological pH) of the globular domain in the artificial-sequence histone H1 with respect to its wild-type counterpart when both globular domains are each in their respective post-translationally unmodified forms or when each globular domain is subjected to plausible post-translational modifications (PTMs), in order to stabilize the electrostatic binding affinity of the artificial-sequence histone H1 (in particular when subjected to PTMs in vivo) to the negatively charged DNA. This effect is possible by virtue of the net electric charge (z) as a function of (i) the side chain and (ii) the ability to undergo PTMs for each amino acid residue placed by the method in the artificial protein sequence and/or those for the amino acid residues displaced by the method from the wild-type protein sequence.

In a more preferred embodiment of the invention, the method entails the application of a set of amino acid substitutions, insertions, and/or deletions to the α₃, α₂, and/or α₁-helix subsequences within the histone H1.0 (also known as histone H1°; H1(0); H5; H1δ; RI H1; or H1 histone family, member 0) protein sequence, to the α₃, α₂, and/or α₁-helix subsequences within the histone H1x (also known as histone H1.10 or H1 histone family, member X) protein sequence, and to the α₃, α₂, and/or α₁-helix subsequences within any respective histone H1.10/H1x ortholog sequence—because said α₃, α₂, and α₁ subsequences correspond to the structural motifs that bind to nucleosomal and/or linker DNA in chromatin.

In an even more preferred embodiment of the invention, the method entails the application of a set of at least one and up to eleven amino acid substitutions at eleven respective specific sites spanning the α₃ amino acid subsequence, the N-terminal region of the α₂ subsequence, and the N-terminal region of the α₁ subsequence, where all three subsequences are within the full sequence of the histone H1.0, histone H1x, and of any respective orthologous protein. This embodiment is even more preferred because amino acid substitutions (as opposed to insertions or deletions) are less likely to disrupt the α-helical geometry and the eleven amino acid substitution sites are those most proximal (within the α-helical motifs) to nucleosomal or linker DNA. The selection of the set of at least one and up to eleven amino acid substitutions includes at least one amino acid substitution in the α₃-helix subsequence. In a further embodiment of the invention, the method comprises at least two more amino acid substitutions, where at least one of the remaining substitutions is made to a α₂, and/or a α₁-helix subsequence within the histone H1.

Therefore, the invention relates in particular to the redesign of the histone H1 α-helical motifs α₃ (most preferred, which binds to both nucleosomal and linker DNA), α₂ (second most preferred, which binds to nucleosomal DNA), and α₁ (third most preferred, which binds to linker DNA), through amino acid sequence modification (by amino acid substitutions, insertions, and/or deletions).

The method for producing artificial histone H1 protein sequences, according to the present invention, is useful for treating and/or preventing senescence, cancer, and/or age-related health conditions.

In this invention, reference is made to the following naturally occurring α-amino acids (aa): alanine (IUPAC one-letter symbol: A, three-letter symbol: Ala), cysteine (C, Cys), aspartic acid (D, Asp), glutamic acid (E, Glu), phenylalanine (F, Phe), glycine (G, Gly), histidine (H, His), isoleucine (I, Ile), lysine (K, Lys), leucine (L, Leu), methionine (M, Met), asparagine (N, Asn), proline (P, Pro), glutamine (Q, Gln), arginine (R, Arg), serine (S, Ser), threonine (T, Thr), valine (V, Val), tryptophan (W, Trp), and tyrosine (Y, Tyr).

Reference is made to amino acid substitutions with the nomenclature X_{W}#X_{A}, where # is the site (identified by counting from the translation initiator Met residue numbered as +1) occupied by a wild-type (wt) amino acid residue X_{W} to be substituted by an amino acid residue X_{A} according to the present invention.

Reference is made to the net electric charge (IUPAC symbol: z) defined as the algebraic sum of the charges present at the surface of a molecule divided by the elementary charge of the proton. In this context, reference is also made in this application to z_{P}, hereby defined as the net electric charge of a molecule at physiological pH (i.e., when pH-7.4). Consequently, a higher z_{P} implies a more positive (or, equivalently, less negative) net electric charge in a molecule at physiological pH. It has to be noted that the application of the set set of amino acid substitutions, insertions and/or deletions according to the present invention entails an increase in the net electric charge of the molecule at physiological pH (z_{P}), it may also modify its net electric charge (z) at other other pH values or ranges.

Reference is made to nucleosomal DNA (also known as core nucleosomal DNA), understood as the DNA that is left-hand wrapped around the histone octamer forming a complex known as the nucleosome core particle (NCP), which is the building block of chromatin.

Reference is made to linker DNA, understood as the DNA that extends in between nucleosome core particles. Importantly for this invention, the phosphate group repeated across the backbone of nucleic acids in particular makes both nucleosomal and linker DNA negatively charged at physiological pH.

Reference is made to the histone H1 (also known as linker histone) protein, which constitutes one of the five major histone protein families necessary for the formation of chromatin in the eukaryotic cell. Specific regions within the histone H1 protein bind to nucleosomal and/or linker DNA, which in turn stabilizes the higher-order constraints on chromatin dynamics.

The histone H1 family comprises a number of variants. These histone H1 variants are encoded by paralog gene families and classified under a phylogeny-based nomenclature. However, they are also grouped according to protein biosynthesis in terms of its relationship with the cell cycle and its tissue specificity.

Reference is made to a major structural motif known as “winged” helix-turn-helix (wHTH) in the form of α₁-β₁-α₂-α₃-β₂-β₃ where the αᵢ motifs are alpha helices and the βⱼ motifs are beta sheets, which characterizes the globular domain of the histone H1 protein. Importantly for this invention, the histone-H1 α₁, α₂, and α₃ helices are motifs with amino acid residues known to be proximal (and thus more likely to bind) to nucleosomal and/or linker DNA.

Reference is also made to post-translational modifications (PTMs), which are covalent and typically (but not necessarily) enzymatic modifications undergone by amino acid residues in proteins following protein biosynthesis.

Histone H1 proteins are subjected to PTMs. Some of the most common PTMs undergone by histone H1 proteins are acetylations and phosphorylations. Lys acetylation is known to lower the otherwise positive electric charge of the Lys residue at physiological pH, in turn decreasing the Lys-mediated electrostatic binding affinity of histone H1 to the negatively charged nucleosomal and linker DNA. The post-translational phosphorylation of amino acid residues such as Ser, Thr, Tyr, and His is also known to decrease the electrostatic DNA binding affinity of the histone H1.

When proximal to DNA-binding regions, the negatively charged Asp and Glu residues may reduce, by electrostatic repulsion, the binding affinity of those regions to nucleosomal and linker DNA.

In general, any wild-type amino acid residue (even a residue of null z, sometimes used itself as a substitute residue in other instances) located in a DNA-binding region of the histone H1 protein may be substituted in the method according to this invention with an amino acid residue such that z_{P} is increased in that region, thereby stabilizing the binding affinity to the negatively charged nucleosomal and linker DNA when that region and/or others in the histone H1 protein are subjected to PTMs in vivo.

When the present invention applies a set of at least one and up to eleven amino acid substitutions to a wild-type histone H1.0 or histone H1x protein sequence, adequate substitute residues include but are not limited to Arg, Ala, Met, Asn, and Pro (the latter being adequate only for substituting an amino acid residue located at the N-terminal site of an α-helical motif).

Since the present invention aims to elicit a significant phenotypic change in the entire multicellular individual in its adult form, the method must target a histone H1 variant that (i) accumulates in terminally differentiated cells and (ii) is synthesized in the whole body of the individual. In other words, the targeted histone H1 variant must be both replication-independent (i.e., synthesized throughout the cell cycle) and somatic.

Importantly, only two histone H1 variants embody both characteristics: the H1.0 (also known as histone H1°; H1(0); H5; H1δ; RI H1; or H1 histone family, member 0) variant, which is common to all multicellular species and the H1x (also known as histone H1.10 or H1 histone family, member X) variant, which is unique to vertebrate species.

Within the histone H1.0/H1x structure, the α₁ helix is 13 amino acid residues long, the α₂ helix is 12 amino acid residues long, and the α₃ helix is 16 amino acid residues long.

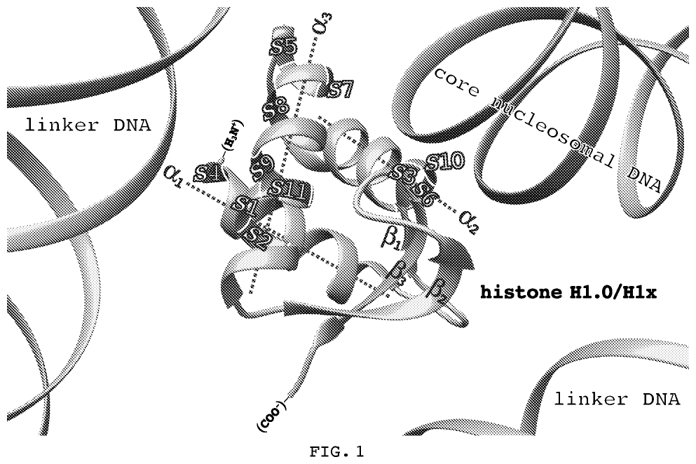

The histone H1.0/H1x α₁ motif (in particular its N-terminal region) binds mainly to linker DNA, the α₂ motif (in particular its N-terminal region) binds mainly to nucleosomal DNA and, importantly, the α₃ binds to both nucleosomal and linker DNA (see FIG. 1).

Amino acid residues in the histone H1.0/H1x α₁, α₂, and/or α₃ motifs can be post-translationally modified. In particular, specific residues can be acetylated or phosphorylated, which entails a decrease in z_{P} for the α-helical motifs, which in turn decreases the electrostatic binding affinity of the histone H1.0/H1x protein to both nucleosomal and/or linker DNA.

The invention presented here corrects for the PTM-dependent decrease in electrostatic DNA binding affinity and/or for an intrinsically low DNA binding affinity—the latter caused mainly by negatively charged amino acid residues—in the wild-type protein without impairing the function of the α-helical motifs within the histone H1.0/H1x protein nor the function of the protein as a whole.

Specifically, this invention claims a method for producing artificial protein sequences and artificial nucleic acid sequences for the histone H1.0 and histone H1x variants and their orthologs, which are useful for the analysis, diagnosis, treatment, and/or prevention of senescence (also known as biological aging) and/or age-related health conditions, such as some types of cancer. This invention also encompasses the artificial protein sequences and artificial nucleic acid sequences produced by the method of the invention.

In the method the application of a set of amino acid substitutions, insertions, and/or deletions to produce artificial histone H1.0 and histone H1x protein sequences (from the three respective wild-type, α-helical subsequences) entails an increase of z_{P} in the artificial-sequence α₃, α₂, and/or α₁ motifs with respect to their wild-type counterparts when the artificial and wild-type motifs are each in their respective post-translationally unmodified forms or when each is subjected to plausible PTMs.

The application of a set of amino acid substitutions only—as opposed to insertions or deletions, because substitutions are more likely to preserve the secondary structure and overall function of the wild-type protein in the artificial-sequence protein derived from it while creating in the artificial-sequence protein a new function for the multicellular individual—applied to a wild-type histone H1.0 or histone H1x informs the most preferred embodiment of this invention.

When the artificial-sequence α₃, α₂, and/or α₁ motifs undergo post-translational modifications in vivo, the set of amino acid substitutions, insertions, and/or deletions applied by the method to produce the artificial-sequence α₃, α₂, and/or α₁ motifs effectively creates a “reservoir” of positive electric charge that stabilizes or enhances the electrostatic binding affinity of the artificial-sequence histone H1.0/H1x protein to the negatively charged nucleosomal and/or linker DNA.

This stabilization or enhancement of the electrostatic binding affinity of the artificial-sequence histone H1.0/H1x protein to nucleosomal and/or linker DNA in turn stabilizes the higher-order constraints on chromatin dynamics in the terminally differentiated cells of the multicellular individual, which in turn translates into significant resistance to senescence and/or to age-related health conditions for the multicellular individual.

For a person skilled in the art, it would be evident that, although the intracellular activity is given by the artificial histone H1 protein, these proteins can be obtained through (i) artificial DNA sequences prepared using any technique available in the state of the art such as genome editing or plasmid systems and (ii) artificial RNA sequences, where all these artificial nucleic acid sequences encoding the artificial protein sequences produced by the claimed method, as well as their complementary reverse in the case of the DNA sequences, are considered within the scope of the present invention.

The non-triviality and high specificity of the set of amino acid substitutions, insertions, and/or deletions—in terms of location (DNA-binding protein regions) and required effect (increase of z in DNA-binding protein regions)—that must in turn be applied to the wild-type sequence of highly specific, functionally well-defined proteins (histone H1.0 and/or histone H1x) constitutes a clear inventive step in this patent application.

The method according to this invention is intended to induce resistance and/or protection against senescence (also known as biological aging) and/or age-related health conditions in multicellular species by producing artificial protein sequences and nucleic acid sequences for the histone H1 variants H1.0 (also known as histone H1°; H1(0); H5; H1δ; RI H1; or H1 histone family, member 0) and H1x (also known as histone H1.10 or H1 histone family, member X), and comprises the steps of:
a. selecting, according to the preferred order of histone H1 variants specified in this invention, a wild-type histone H1.0 protein sequence, or a wild-type histone H1x protein sequence, or the sequence of a respective protein ortholog in the species of interest;
b. within the sequence selected in step a, recognizing the regions or individual sites in the globular domain or in its α-helical motifs (for the latter, see steps b.2, b.3, and b.4) of the protein that are most proximal to DNA—using, if necessary, the X. laevis histone H1 protein 3D structure contained in the publicly available PDB data file 5NL0 as a structural homology guide—and/or recognizing the regions or individual sites in the globular domain of the protein known to bind to DNA in the species of interest. In a more preferred realization of this step:
   b.2. depending on the variant of the wild-type histone H1 protein sequence selected (i.e., H1.0 or H1x) in step a, recognizing the first (counting from N- to C-terminus) α-helical motif α₁ by using as a phylogenetic homology guide the amino acid sequence of SEQ. ID NO. 1 if the wild-type histone variant is H1.0 or the amino acid sequence of SEQ. ID NO. 4 if the wild-type histone variant is H1x;
   b.3. depending on the variant of the wild-type histone H1 protein sequence selected (i.e., H1.0 or H1x) in step a, recognizing the second (counting from N- to C-terminus) α-helical motif α₂ by using as a phylogenetic homology guide the amino acid sequence of SEQ. ID NO. 2 if the wild-type histone variant is H1.0 or the amino acid sequence of SEQ. ID NO. 5 if the wild-type histone variant is H1x;
   b.4. depending on the variant of the wild-type histone H1 protein sequence selected (i.e., H1.0 or H1x) in step a, recognizing the third (counting from N- to C-terminus) α-helical motif α₃ by using as a phylogenetic homology guide the amino acid sequence of SEQ. ID NO. 3 if the wild-type histone variant is H1.0 or the amino acid sequence of SEQ. ID NO. 6 if the wild-type histone variant is H1x, and also, in an even more preferred realization of this step:
   b.5. identifying the following eleven amino acid substitution sites (S1, . . . , S11) defined according to their relative position (counting from N- to C-terminus) within each α-helix motif as follows:

| substitution site | α-helix motif | position # (N- to C- ) |
|---|---|---|
| S1 | α₃ | 12 |
| S2 | α₃ | 13 |
| S3 | α₂ | 1 |
| S4 | α₁ | 1 |
| S5 | α₃ | 1 |
| S6 | α₂ | 3 |
| S7 | α₃ | 3 |
| S8 | α₃ | 5 |
| S9 | α₃ | 9 |
| S10 | α₂ | 2 |
| S11 | α₃ | 11; |

b.6. mapping the amino acid substitution sites (S1, . . . , S11) identified in step b.5 into the wild-type protein sequence selected in step a with respect to its three α-helix subsequences recognized in steps b.2, b.3, and b.4;
c. applying a set of amino acid substitutions, insertions, and/or deletions to one or more of the amino acid subsequences corresponding to the regions or sites (for individual sites the subsequence length is equal to one amino acid residue) recognized only in step b or in steps b.2 to b.4 such that modifications do not alter the α-helical structures and such that the respective net electric charge (z) associated to each resulting modified amino acid subsequence is greater, particularly at physiological pH, than the net electric charge (z) associated to its wild-type amino acid subsequence counterpart when the modified amino acid subsequence and the wild-type amino acid subsequence are each in their respective post-translationally unmodified forms or when each is subjected to a respective combination of post-translational modifications (PTMs). Or, alternatively, if steps b.5 and b.6 were also made:
   c.2. applying a set of amino acid substitutions in a sequential yet not necessarily comprehensive manner—where the set of amino acid substitutions is not only applied to the wild-type protein sequence in a sequential manner but also tested experimentally/clinically in a sequential manner, i.e., experimentally/clinically testing the amino acid substitutions specified by the present invention only one or two at a time (preferred in the context of artificial-sequence proteins for humans, because the application of a minimal number k<11 of amino acid substitutions, provided the k amino acid substitutions elicit the desired phenotypic change, turn renders the remaining k+1, . . . , 11 amino acid substitutions superfluous), and where any substitute amino acid residue identical to the wild-type amino acid residue it is supposed to replace at any site among (S1, . . . , S11) simply implies no action taken and the set of amino acid substitutions to be reduced in one element for each such case (preferred for simplicity and for keeping the amino acid substitutions applied as few as possible provided they elicit the desired phenotypic change as explained previously)—at the sites (S1, . . . , S11), which are now mapped into the wild-type protein sequence, according to the following criteria:

| substitution site | if | wt residue is | substitute with |
|---|---|---|---|
| S1 | | (K ∨ S ∨ T) | R |
| S2 | | (S ∨ T ∨ M ∨ L) | R |
| S3 | | (K ∨ L) | R |

¬: logical NOT;
∨: logical OR;

| substitution site | if | wt residue is | substitute with |
|---|---|---|---|
| S3 | | (S ∨ T) | P |
| S4 | | (S ∨ T) | P |
| S5 | | K | M |
| S5 | | (S ∨ T) | N |
| S6 | | (S ∨ T) | A |
| S7 | | (D ∨ E) | N |
| S8 | | (S ∨ T ∨ Y) | R |
| S9 | | (S ∨ T) | A |
| S10 | | Y | R |
| S11 | | ¬R | R |

¬: logical NOT;
∨: logical OR;

c.3. verifying that the set of amino acid substitutions applied in step c.2 satisfies the condition of increased z_{P} by estimating z at physiological pH for each modified α-helical motif and comparing it to the z estimate at physiological pH for its wild-type counterpart when the artificial-sequence (i.e., modified) α-helical motif and the wild-type α-helical motif are each in their respective post-translationally unmodified forms or when each is subjected to plausible PTMs;
d. optimizing (if necessary for technical and/or biological reasons) the set of amino acid substitutions applied in step c or in step c.2 by using alternative substitute residues (i.e., other than those suggested in this method) with the same rationale of increased z_{P} in the artificial-sequence histone H1.0/H1x while preserving the secondary structure and overall function of the wild-type histone H1.0/H1x, thereby allowing in the artificial-sequence histone H1.0/H1x the creation of a novel function for the multicellular individual on top of the regular function inherited from its wild-type protein counterpart;
e. consolidating the set of amino acid substitutions, insertions, and/or deletions determined by steps c, c.2, c.3, and d into the wild-type histone H1.0, histone H1x, or respective orthologous protein sequence selected in step a, thereby producing the complete artificial protein sequence—where the applied set of amino acid substitutions, insertions, and/or deletions effectively creates a “reservoir” of positive electric charge in the artificial-sequence histone H1.0/H1x protein produced, thereby stabilizing or enhancing its electrostatic binding affinity (with respect to its wild-type counterpart) to DNA; and
f. optionally, producing—by virtue of the degeneracy of the genetic code and, if necessary, under the constraints imposed by the species of interest (e.g., codon usage bias) and experimental technique (e.g., CRISPR/Cas sgRNA design)—an artificial nucleic acid sequence that encodes the artificial protein sequence produced in step e.
   Steps b.2, b.3, and b.4 are preferred because because (i) the histone H1.0/H1x α-helical motifs are specifically known to bind to nucleosomal and/or linker DNA and (ii) the condition of increased net electric charge (z) in the artificial α-helical motifs with respect to their wild-type counterparts creates a “reservoir” of positive electric charge in the artificial α-helical motifs. Steps b.5, b.6, c.2, and c.3 are even more preferred because (i) in the histone H1.0/H1x the α₃ motif is known to bind to both nucleosomal and linker DNA (most preferred), the α₂ motif is known to bind to nucleosomal DNA (second most preferred), and the α₁ motif is known to bind to linker DNA (third most preferred), (ii) the eleven amino acid substitution sites (S1, . . . , S11) are highly specific and most proximal (within the α-helical motifs) to nucleosomal or linker DNA, and (iii) the substitute amino acid residues Arg, Ala, Met, Asn, and Pro are able to create, when replacing specific amino acid residues at specific sites, a “reservoir” of positive electric charge in the artificial α-helical motifs as detailed previously.

The present invention encompasses artificial histone H1.0 or H1x protein sequences for inducing resistance and/or protection against senescence, and/or age-related health conditions—which include but not are not limited to age-related cancer, atherosclerosis and cardiovascular disease, arthritis, cataracts, osteoporosis, type-2 diabetes, hypertension, Alzheimer's disease, benign prostate hyperplasia, hearing disability, age-related macular degeneration, neurodegenerative diseases, degenerative diseases, immune senescence diseases, skin aging, and skin wrinkles—where the artificial protein sequence contains a set of at least one amino acid substitutions, insertions, and/or deletions to the DNA-binding site of the histone H1.0 or H1x proteins in the α-helical regions, where the substitutions, insertions, and/or deletions do not alter the structure of the α-helical motifs and also entail an increase in the net electric charge (z) of the resulting artificial-sequence protein. The net electric charge (z) is estimated particularly at physiological pH.

The DNA binding sites are located in the first, second, and/or third (counting from N- to C-terminus) α-helices of the histone H1.0 and histone H1x proteins.

The amino acid sequence that corresponds to the first α-helix, denoted by α₁, of the wild-type histone H1 protein counterpart is identical or homologous to SEQ. ID No. 1 if the wild-type histone variant is H1.0 or to the SEQ. ID No. 4 if the wild-type histone variant is H1x.

The amino acid sequence that corresponds to the second α-helix, denoted by α₂, of the wild-type histone H1 protein counterpart is identical or homologous to SEQ. ID No. 2 if the wild-type histone variant is H1.0 or to SEQ. ID No. 5 if the wild-type histone variant is H1x.

The amino acid sequence that corresponds to the third α-helix, denoted by α₂, of the wild-type histone H1 protein counterpart is identical or homologous to SEQ. ID No. 3 if the wild-type histone variant is H1.0 or to SEQ. ID No. 6 if the wild-type histone variant is H1x.

In an embodiment of the invention, the set of amino acid modifications corresponds to at least one to up to eleven amino acid substitutions within the binding site in the α-helical motif.

In a further embodiment of the invention, the eleven amino acid substitution sites α₁ to α₁₁ comprise at least one substitution for each of the first, second, and third α-helical motifs selected from: (S1,α₃,12), (S2,α₃,13), (S3,α₂,1), (S4,α₁,1), (S5,α₃,1), (S6,α₂,3), (S7,α₃,3), (S8,α₃,5), (S9,α₃,9), (S10,α₂,2) and (S11,α₃,11); where each triplet shows the substitution site, the α-helical motif and the relative position (counting from N- to C-terminus); where the substitute amino acid residue are selected from alanine, methionine, leucine and arginine, for any substitution site or proline for substitution sites α₃ and/or α₄.

For a proper z_{P} comparison between artificial and wild-type protein sequences, the same dissociation-constant data—i.e., the same set of pKₐ values for the α-carboxylic acid group, α-ammonium group, and side chain group (if applicable) of each amino acid residue—must be used as calculation base.

The artificial-sequence histone H1.0 and histone H1x proteins according to the present invention, when synthesized by engineered cells (e.g., via genome editing or synthetic mRNA delivery) or administered extrinsically to cells (if extracellular histone H1 cytotoxicity can be countered) so that in treated multicellular individuals the artificial-sequence proteins reach abundance levels comparable to those of their wild-type protein counterparts in untreated individuals, confer the treated individuals significant resistance to senescence and/or age-related health conditions.

The artificial histone H1.0 and histone H1x protein sequences, the synthetic or recombinant nucleic acid sequences encoding said artificial protein sequences, and the methods for producing such sequences according to this invention are useful for analyzing and/or diagnosing senescence and/or age-related health conditions in multicellular species.

The artificial histone H1.0 and histone H1x protein sequences according to this invention are useful for inducing resistance and/or protection against senescence and age-related health conditions in multicellular species. Said resistance and/or protection includes, but is not limited to, the analysis, diagnosis, treatment and/or prevention of senescence and/or other age-related health conditions, such as certain types of cancer.

The artificial histone H1.0 and histone H1x protein sequences—and the synthetic or recombinant nucleic acid sequences encoding them—according to this invention can be used on biomedical, cosmetic, industrial, and agricultural applications.

The method of the invention was tested in vivo on a simple organism, such as C. elegans, in order to verify its effectiveness, and as can be seen in detail in examples 1 and 11, a C. elegans organism was obtained featuring only three amino acid substitutions in the sequence of its histone H1.X protein (ortholog of the human histone H1.0) and displating great resistance to senescence, which translates to a very significant increase in the survival rate. So much so that by day 14 only 50% of the wild type survived and 100% of the mutants (hil-1 gene), and by day 24 there were no wild-type left alive, while the worms which synthesized the mutant histone H1.X protein developed in accordance with the method of the present invention, showing a survival rate above 98%. Based on the results, where 98% of the mutants (subjected to only 3 amino acid substitutions in their histone H1.X protein) are still alive, whereas 100% of the wild-type individuals are dead.

For a person skilled in the art, it would be evident that, although the intracellular activity is given by the artificial histone H1 protein, these proteins can be obtained through (i) artificial DNA sequences prepared using any technique available in the state of the art such as genome editing or plasmid systems and (ii) artificial RNA sequences, where all these artificial nucleic acid sequences encoding the artificial protein sequences produced by the claimed method, as well as their complementary reverse in the case of the DNA sequences, are considered within the scope of the present invention.

### Examples

The following examples are intended to illustrate the present invention and they cannot be used for limiting its scope.

Example 1: Application of the most preferred embodiment of the method for producing an artificial protein sequence for the somatic, replication-independent histone H1 variant in the model organism Caenorhabditis elegans.
The only replication-independent and somatic histone H1 variant (ortholog of the human histone H1.0) in C. elegans is the histone H1.X protein (NCBI ID: NP_506680.1), SEQ.ID No. 7:

>NP_506680.1 Histone H1.X [Caenorhabditis elegans]
MTTSLIHMANHLDASTEEISLNYVLLGHPHHERAQHHPSYMDMIKGAIQA
IDNGTGSSKAAILKYIAQNYHVGENLPKVNNHLRSVLKKAVDSGDIEQTR
GHGATGSFRMGKECEKNLQVGIPVQTKPMLMLKEVRQKLENISKAEKTKP
STSSMSTNKKGKPISTMKKRGVMSKKRSSKNKMAPKAKSHGLKKKGPATK
SSGLVHKAAGAKNEAAPTTKMELRTGTRKSYC

Using the sequences of the human histone H1.0 α-helical motifs provided in the method as phylogenetic homology guides (SEQ. ID Nos. 1, 2, and 3), the respective subsequences corresponding to the α₁, α₂, and α₃ motifs were recognized in the C. elegans wild-type histone H1.X protein sequence:

| | |
|---|---|
| C. elegans H1.X | 39 SYMDMIKGAIQAI DNGT GSS KAAILKYIAQNY HVGENLP KVNNHLRSVLKKAVDS 93 |
| H. sapiens H1.0 | 27 KYSDMIVAAIQAE KNRA GSS RQSIQKYIKSHY KVGE NADSQIKLSIKRLVTT 78 |
| wHTH motif | α₁ α₂ α₃ |

Next, the predefined amino acid substitution sites (S1, . . . , S11) were mapped into the C. elegans wild-type histone H1.X sequence:

| | |
|---|---|
| substitution site S | 4 3 106 5 7 8 9 11 1 2 |
| | ↓ ↓ ↓ ↓ ↓ ↓ ↓ ↓ ↓ ↓ ↓ |
| | 39 596061 788082 86 888990 |
| C. elegans H1.X | 39 SYMDMIKGAIQAI DNGT GSS KAAILKYIAQNY HVGENLP KVNNHLRSVLKKAVDS 93 |
| wHTH motif | α₁ α₂ α₃ |

In this example only one of the possible embodiments of this invention were produced by applying the method to the wild-type C. elegans histone H1.X reference sequence up to the substitution site S4:

| site | position [#] | wt residue | substitute residue | aa substitution |
|---|---|---|---|---|
| S1 ✓ | 89 | K | R | K89R |
| S2 [symbol] | 90 | A | n/a | n/a |
| S3 ✓ | 59 | K | R | K59R |
| S4 ✓ | 39 | S | P | S39P |

n/a: not applicable in the method

Since the amino acid substitutions K89R, K59R, and S39P are encompassed by the helical motifs α₃, α₂, and α₁ respectively, it was next verified that the estimated z_{P} of each artificial-sequence α-helical motif (substitute amino acid residues underlined) is greater than that of its wild-type C. elegans counterpart—when both are post-translationally unmodified or when both are subjected to plausible PTMs (online PTM prediction programs can be useful for this step):

| α-helix sequence | seq. type | zₚ (est.) |
|---|---|---|
| α₃ (no PTMs) | | |
| (H₃N⁺)-KVNNHLRSVLKKAVDS-(coo⁻) | wild-type | +2.193 |
| (H₃N⁺)-KVNNHLRSVLKRAVDS-(coo⁻) | artificial | +2.197 > +2.193 ✓ |
| α₃ (with plausible PTMs) | | |
| (H₃N⁺)-KVNNHLR(pS)VL(K-ac)KAVDS-(coo⁻) | wild-type | −0.563 |
| (H₃N⁺)-KVNNHLR(pS)VL(K-ac)RAVDS-(coo⁻) | artificial | −0.559 > −0.563 ✓ |
| α₂ (no PTMs) | | |
| (H₃N⁺)-KAAILKYIAQNY-(coo⁻) | wild-type | +1.146 |
| (H₃N⁺)-RAAILKYIAQNY-(coo⁻) | artificial | +1.178 > +1.146 ✓ |
| α₂ (with plausible PTMs ) | | |
| (H₃N⁺)-KAAIL(K-ac)YIAQNY-(coo⁻) | wild-type | +0.150 |
| (H₃N⁺)-RAAIL(K-ac)YIAQNY-(coo⁻) | artificial | +0.182 > +0.150 ✓ |
| α₁ (no PTMs) | | |
| (H₃N⁺)-SYMDMIKGAIQAI-(coo⁻) | wild-type | −0.783 |
| (H₃N⁺)-PYMDMIKGAIQAI-(coo⁻) | artificial | −0.106 > −0.783 ✓ |
| α₁ (with plausible PTMs ) | | |
| (H₃N⁺)-(pS)YMD(oM)I(K-ac)GAIQAI-(coo⁻) | wild-type | −3.539 |
| (H₃N⁺)-PYMD(oM)I(K-ac)GAIQAT-(coo⁻) | artificial | −1.102 > −3.539 ✓ |

(K-ac): acetylated Lys;
(oM): oxidized Met;
(pS): phosphorylated Ser

One of the possible artificial protein sequences was finally produced (SEQ. ID No. 8, as claimed in this invention), which is defined by the set of amino acid substitutions {K89R, K59R, S39P} when applied to the wild-type C. elegans histone H1.X reference sequence (substitute amino acid residues underlined):

>example-01 artificial-sequence histone H1.X for
C. elegans
MTTSLIHMANHLDASTEEISLNYVLLGHPHHERAQHHPPYMDMIKGAIQA
IDNGTGSSRAAILKYIAQNYHVGENLPKVNNHLRSVLKRAVDSGDIEQTR
GHGATGSFRMGKECEKNLQVGIPVQTKPMLMLKEVRQKLENISKAEKTKP
STSSMSTNKKGKPISTMKKRGVMSKKRSSKNKMAPKAKSHGLKKKGPATK
SSGLVHKAAGAKNEAAPTTKMELRTGTRKSYC

Examples 2-3: Application of the most preferred embodiment of the method for producing two artificial sequences for the mouse (Mus musculus) histone H1.0 protein.
The reference sequence for the mouse histone H1.0 protein (NCBI ID: NP 032223.2), SEQ. ID No. 9, is the following:

>NP_032223.2 histone H1.0 [Mus musculus]
MTENSTSAPAAKPKRAKASKKSTDHPKYSDMIVAAIQAEKNRAGSSRQS
IQKYIKSHYKVGENADSQIKLSIKRLVTTGVLKQTKGVGASGSFRLAKG
DEPKRSVAFKKTKKEVKKVATPKKAAKPKKAASKAPSKKPKATPVKKAK
KKPAATPKKAKKPKVVKVKPVKASKPKKAKTVKPKAKSSAKRASKKK

Using the sequences of the human histone H1.0 α-helical motifs provided in the method as phylogenetic homology guides (SEQ. ID Nos. 1, 2, and 3), the respective subsequences corresponding to the α₁, α₂, and α₃ motifs were recognized in the M. musculus wild-type histone H1.0 protein sequence:

| | |
|---|---|
| M.musculus H1.0 | 27 KYSDMIVAAIQAE KNRA GSS RQSIQKYIKSHY KVGE NADSQIKLSIKRLVTT 78 |
| H. sapiens H1.0 | 27 KYSDMIVAAIQAE KNRA GSS RQSIQKYIKSHY KVGE NADSQIKLSIKRLVTT 78 |
| wHTH motif | α₁ α₂ α₃ |

Next, the predefined amino acid substitution sites (S1, . . . , S11) were mapped into the M. musculus wild-type histone H1.0 sequence:

| | |
|---|---|
| substitution site S | 4 3 10 6 5 7 8 9 11 1 2 |
| | ↓ ↓ ↓ ↓ ↓ ↓ ↓ ↓ ↓ ↓ ↓ |
| | 27 47 48 49 63 65 67 71 73 74 75 |
| M. musculus H1.0 | 27 KYSDMIVAAIQAE KNRA GSS RQSIQKYIKSHY KVGE NADSQIKLSIKRLVTT 78 |
| wHTH motif | α₁ α₂ α₃ |

In example 2 the method was to produce a “conservative” artificial protein sequence (in terms of departure from its wild-type counterpart) and thus it was produced by applying the method to the wild-type mouse histone H1.0 reference sequence only up to the substitution site S2:

| site | position [#] | wt residue | substitute residue | aa substitution |
|---|---|---|---|---|
| S1 [symbol] | 74 | R | n/a | n/a |
| S2 ✓ | 75 | L | R | L75R |

n/a: not applicable in the method

Since the amino acid substitution L75R is encompassed by the helical motif α₃, it was next verified that the estimated z_{P} of the artificial-sequence α₃ helix (substitute amino acid residue underlined) is greater than that of its wild-type mouse counterpart—when both are post-translationally unmodified or when both are subjected to plausible PTMs (online PTM prediction programs can be useful for this step):

| α-helix sequence | seq. type | zp (est.) |
|---|---|---|
| α₃ (no PTMs) | | |
| (H₃N⁺)-NADSQIKLSIKRLVTT-(coo⁻) | wild-type | +1.391 |
| (H₃N⁺)-NADSQIKLSIKRRVTT-(coo⁻) | artificial | +2.391 > +1.391 ✓ |
| α₃ (with plausible PTMs) | | |
| (H₃N⁺)-NAD(pS)QI(K-ac)LSIKRLVTT-(coo⁻) | wild-type | -1.365 |
| (H₃N⁺)-NAD(pS)QI(K-ac)LSIKRRVTT-(coo⁻) | artificial | -0.365 > -1.365 ✓ |

(PS): phosphorylated Ser;
(K-ac): acetylated Lys

An artificial protein sequence was finally produced (SEQ. ID No. 10, as claimed in this invention), which is defined by the set of amino acid substitutions {L75R} when applied to the wild-type mouse histone H1.0 reference sequence (substitute amino acid residue underlined):
example-02 artificial-sequence histone H1.0 for M. musculus

MTENSTSAPAAKPKRAKASKKSTDHPKYSDMIVAAIQAEKNRAGSSRQS
IQKYIKSHYKVGENADSQIKLSIKRRVTTGVLKQTKGVGASGSFRLAKG
DEPKRSVAFKKTKKEVKKVATPKKAAKPKKAASKAPSKKPKATPVKKAK
KKPAATPKKAKKPKVVKVKPVKASKPKKAKTVKPKAKSSAKRASKKK

In example 3 the method was to produce a less “conservative” artificial protein sequence (in terms of departure from its wild-type counterpart) and thus it was produced by applying the method to the wild-type mouse histone H1.0 reference sequence up to the substitution site S7:

| site | position [#] | wt residue | substitute residue | aa substitution |
|---|---|---|---|---|
| S1 [symbol] | 74 | R | n/a | n/a |
| S2 ✓ | 75 | L | R | L75R |
| S3 [symbol] | 47 | R | n/a | n/a |
| S4 [symbol] | 27 | K | n/a | n/a |
| S5 [symbol] | 63 | N | n/a | n/a |
| S6 ✓ | 49 | S | A | S49A |
| S7 ✓ | 65 | D | N | D65N |

n/a: not applicable in the method

Since the amino acid substitutions L75R and D65N are encompassed by the helical motif α₃ and the amino acid substitution S49A is encompassed by the helical motif α₂, it was next verified that the estimated z_{P} of each of the artificial-sequence α₃ and α₂ motifs (substitute amino acid residues underlined) is greater than that of its wild-type mouse counterpart—when both are post-translationally unmodified or when both are subjected to plausible PTMs (online PTM prediction programs can be useful for this step):

| α-helix sequence | seq. type | zₚ (est.) |
|---|---|---|
| α₃ (no PTMs) | | |
| (H₃N⁺)-NADSQIKLSIKRLVTT-(coo⁻) | wild-type | +1.391 |
| (H₃N⁺)-NANSQIKLSIKRRVTT-(coo⁻) | artificial | +3.390 > +1.391 ✓ |
| α₃ (with plausible PTMs) | | |
| (H₃N⁺)-NAD(pS)QI(K-ac)LSIKRLVTT-(coo⁻) | wild-type | -1.365 |
| (H₃N⁺)-NAN(pS)QI(K-ac)LSIKRRVTT-(coo⁻) | artificial | +0.634 > -1.365 ✓ |
| α₂ (no PTMs) | | |
| (H₃N⁺)-RQSIQKYIKSHY-(coo⁻) | wild-type | +2.219 |
| (H₃N⁺)-RQAIQKYIKSHY-(coo⁻) | artificial | +2.219 = +2.219 |
| α₂ (with plausible PTMs) | | |
| (H₃N⁺)-RQ(pS)IQ(K-ac)YIKSHY-(coo⁻) | wild-type | -0.536 |
| (H₃N⁺)-RQAIQ(K-ac)YIKSHY-(coo⁻) | artificial | +1.223 > -0.536 ✓ |

(K-ac): acetylated Lys;
(PS): phosphorylated Ser

An artificial protein sequence was finally produced (SEQ. ID No. 11, as claimed in this invention), which is defined by the set of amino acid substitutions {L75R, S49A, D6511} when applied to the wild-type mouse histone H1.0 reference sequence (substitute amino acid residues underlined):
>example-03 artificial-sequence histone H1.0 for M. musculus

MTENSTSAPAAKPKRAKASKKSTDHPKYSDMIVAAIQAEKNRAGSSRQA
IQKYIKSHYKVGENANSQIKLSIKRRVTTGVLKQTKGVGASGSFRLAKG
DEPKRSVAFKKTKKEVKKVATPKKAAKPKKAASKAPSKKPKATPVKKAK
KKPAATPKKAKKPKVVKVKPVKASKPKKAKTVKPKAKSSAKRASKKK

Examples 4-5: Application of the most preferred embodiment of the method for producing one artificial sequence for the human histone H1.0 protein and one artificial sequence for the human histone H1x protein.
The reference sequence for the human histone H1.0 protein (NCBI ID: NP 005309.1), SEQ. ID No. 12, is the following:

>NP_005309.1 histone H1.0 [Homo sapiens]
MTENSTSAPAAKPKRAKASKKSTDHPKYSDMIVAAIQAEKNRAGSSRQS
IQKYIKSHYKVGENADSQIKLSIKRLVTTGVLKQTKGVGASGSFRLAKS
DEPKKSVAFKKTKKEIKKVATPKKASKPKKAASKAPTKKPKATPVKKAK
KKLAATPKKAKKPKTVKAKPVKASKPKKAKPVKPKAKSSAKRAGKKK

The phylogenetic homology guides provided in the method correspond to the respective α-helical motifs from the human histone H1.0 and H1x variants, thus recognizing the respective subsequences corresponding to the three α-helical motifs in the wild-type histone H1.0, using the sequences SEQ. ID Nos. 1, 2, and 3, and mapping the predefined amino acid substitution sites into its sequence were trivial steps:

| | |
|---|---|
| substitution site S | 4 3 10 6 5 7 8 9 11 1 2 |
| | ↓ ↓ ↓ ↓ ↓ ↓ ↓ ↓ ↓ ↓ ↓ |
| | 27 47 48 49 63 65 67 71 73 74 75 |
| H.sapiens H1.0 | 27 KYSDMIVAAIQAE KNRA GSS RQSIQKYIKSHY KVGE NADSQIKLSIKRLVTT 78 |
| wHTH motif | α₁ α₂ α₃ |

In example 4 the method was to produce a “conservative” artificial protein sequence (in terms of departure from its wild-type counterpart) because it is for use in humans. Thus, the artificial protein sequence was produced by applying the method to the wild-type human histone H1.0 reference sequence only up to the substitution site S2:

| site | position [#] | wt residue | substitute residue | aa substitution |
|---|---|---|---|---|
| S1 [symbol] | 74 | R | n/a | n/a |
| S2 ✓ | 75 | L | R | L75R |

n/a: not applicable in the method

Since the amino acid substitution L75R is encompassed by the helical motif α₃, it was next verified that the estimated z_{P} of the artificial-sequence α₃ helix (substitute amino acid residue underlined) is greater than that of its wild-type human counterpart—when both are post-translationally unmodified or when both are subjected to plausible PTMs (online PTM prediction programs can be useful for this step):

| α-helix sequence | seq. type | zₚ (est.) |
|---|---|---|
| α₃ (no PTMs) | | |
| (H₃N⁺)-NADSQIKLSIKRLVT T-(coo⁻) | wild-type | +1.391 |
| (H₃N⁺)-NADSQIKLSIKRRVT T-(coo⁻) | artificial | +2.391 > +1.391 ✓ |
| α₃ (with plausible PTMs) | | |
| (H₃N⁺)-NAD(pS)QI(K-ac)LSIKRLVTT-(coo⁻) | wild-type | −1.365 |
| (H₃N⁺)-NAD(pS)QI(K-ac)LSIKRRVTT-(coo⁻) | artificial | −0.365 > −1.365 ✓ |

(PS): phosphorylated
Ser; (K-ac): acetylated Lys

An artificial protein sequence was finally produced (SEQ. ID No. 13, as claimed in this invention), which is defined by the set of amino acid substitutions {L75R} when applied to the wild-type human histone H1.0 reference sequence (substitute amino acid residue underlined):
>example-04 artificial-sequence histone H1.0 for H. sapiens

MTENSTSAPAAKPKRAKASKKSTDHPKYSDMIVAAIQAEKNRAGSSRQS
IQKYIKSHYKVGENADSQIKLSIKRRVTTGVLKQTKGVGASGSFRLAKS
DEPKKSVAFKKTKKEIKKVATPKKASKPKKAASKAPTKKPKATPVKKAK
KKLAATPKKAKKPKTVKAKPVKASKPKKAKPVKPKAKSSAKRAGKKK

The reference sequence for the human histone H1x protein (NCBI ID: NP 006017.1), SEQ. ID No. 14, is the following:
>NP_006017.1 histone H1x [Homo sapiens]

MSVELEEALPVTTAEGMAKKVTKAGGSAALSPSKKRKNSKKKNQPGKYS
QLVVETIRRLGERNGSSLAKIYTEAKKVPWFDQQNGRTYLKYSIKALVQ
NDTLLQVKGTGANGSFKLNRKKLEGGGERRGAPAAATAPAPTAHKAKKA
APGAAGSRRADKKPARGQKPEQRSHKKGAGAKKDKGGKAKKTAAAGGKK
VKKAAKPSVPKVPKGRK

The phylogenetic homology guides provided in the method correspond to the respective α-helical motifs from the human histone H1.0 and H1x variants, thus recognizing the respective subsequences corresponding to the three α-helical motifs in the wild-type histone H1x, using the sequences SEQ. ID Nos. 4, 5 and 6, and mapping the predefined amino acid substitution sites into its sequence were trivial steps:

| | |
|---|---|
| substitution site s | 4 3 10 6 5 7 8 9 11 1 2 |
| | ↓ ↓ ↓ ↓ ↓ ↓ ↓ ↓ ↓ ↓ ↓ |
| | 47 67 68 69 84 86 88 92 94 95 96 |
| H. sapiens Hlx | 47 KYSQLVVETIRRL GERN GSS LAKIYTEAKKVP WFDQQ NGRTYLKYSIKALVQN 99 |
| wHTH motif | α₁ α₂ α₃ |

In example 5 the method was to produce a “conservative” artificial protein sequence (in terms of departure from its wild-type counterpart) because it is for use in humans. Thus, the artificial protein sequence was produced by applying the method to the wild-type human histone H1x reference sequence only up to the substitution site S2:

| site | position [#] | wt residue | substitute residue | aa substitution |
|---|---|---|---|---|
| S1 ✓ | 95 | A | R | A95R |
| S2 ✓ | 96 | L | R | L96R |

Since the amino acid substitution A95R and L96R are encompassed by the helical motif α₃, it was next verified that the estimated z_{P} of the artificial-sequence α₃ helix (substitute amino acid residues underlined) is greater than that of its wild-type human counterpart—when both are post-translationally unmodified or when both are subjected to plausible PTMs (online PTM prediction programs can be useful for this step):

| α₃-helix subsequence | subseq. type | zₚ (est.) |
|---|---|---|
| α₃ (no PTMs) | | |
| (H₃N⁺)-NGRTYLKYSIKALVQN-(coo⁻) | wild-type | +2.383 |
| (H₃N⁺)-NGRTYLKYSIKRRVQN-(coo⁻) | artificial | +4.383 > +2.383 ✓ |
| α₃ (with plausible PTM) | | |
| (H₃N⁺)-NGR(pT)YL(K-ac)YSIKALVQN-(coo⁻) | wild-type | −0.373 |
| (H₃N⁺)-NGR(pT)YL(K-ac)YSIKRRVQN-(coo⁻) | artificial | +1.627 > −0.373 ✓ |

(PT): phosphorylated Thr;
(K-ac): acetylated Lys

An artificial protein sequence was finally produced (SEQ. ID No. 15, as claimed in this invention), which is defined by the set of amino acid substitutions {A95R, L96R} when applied to the wild-type human histone H1x reference sequence (substitute amino residues underlined):
>example-05 artificial-sequence histone H1x for H. sapiens

MSVELEEALPVTTAEGMAKKVTKAGGSAALSPSKKRKNSKKKNQPGKYS
QLVVETIRRLGERNGSSLAKIYTEAKKVPWFDQQNGRTYLKYSIKRRVQ
NDTLLQVKGTGANGSFKLNRKKLEGGGERRGAPAAATAPAPTAHKAKKA
APGAAGSRRADKKPARGQKPEQRSHKKGAGAKKDKGGKAKKTAAAGGKK
VKKAAKPSVPKVPKGRK

Examples 6-10: Since a protein ortholog of the human histone H1.0 can be found in all multicellular species and a protein ortholog of the human histone H1x can be found in all vertebrate species, the method informing the present invention can produce artificial histone H1.0 sequences for any multicellular species and artificial histone H1x sequences for any vertebrate species. Five examples of artificial histone H1.0/H1x protein sequences produced with the method claimed in this invention, using its most preferred steps, for different species are shown in TABLE 1.

TABLE 1

| | | | | wt histone H1.0/H1x-orthologous sequence |
|---|---|---|---|---|
| | | use of the method by applying | | [SEQ. ID.] thus producing artificial |
| example | species | the set of aa substitutions | to the | sequence {SEQ. ID} for (H1 variant) |
| 6 | Rattus norvegicus | {L75R} | | [16] {17} (H1.0) |
| 7 | Rattus norvegicus | {A94R, L95R} | | [18] {19} (H1x) |
| 8 | Nothobranchius furzeri | {L73R, S47A, D63N} | | [20] {21} (H1.0) |
| 9 | Drosophila melanogaster | {S96R, L68R, K85M} | | [22] {23} (H1.0) |
| 10 | Arabidopsis thaliana | {S76R, S64N, T68R, Y47R} | | [24] {25} (H1.0) |

Example 11: In vivo testing of the method for conferring C. elegans resistance to senescence.
a. The artificial sequence for the C. elegans histone H1.X protein (amino acid substitutions underlined) produced in example 1 (SEQ. ID No. 8) is:

>example-01 artificial-sequence histone
H1.X for C. elegans
MTTSLIHMANHLDASTEEISLNYVLLGHPHHERAQHHPPYMDMIKGAIQ
AIDNGTGSSRAAILKYIAQNYHVGENLPKVNNHLRSVLKRAVDSGDIEQ
TRGHGATGSFRMGKECEKNLQVGIPVQTKPMLMLKEVRQKLENISKAEK
TKPSTSSMSTNKKGKPISTMKKRGVMSKKRSSKNKMAPKAKSHGLKKKG
PATKSSGLVHKAAGAKNEAAPTTKMELRTGTRKSYC

b. The wild-type C. elegans histone H1.X protein is encoded by the hil-1 gene. Thus, it was necessary to edit the hil-1 gene in the wild-type C. elegans genome (with the CRISPR/Cas genome-editing technique) so that the resulting mutant hil-1 gene encodes the artificial protein sequence shown in step a.
c. The CRISPR/Cas genome editing in the wild-type C. elegans (strain N2) was carried out successfully. The mutant hil-1 allele obtained was fluorescently tagged, then outcrossed to N2 ten times and found to be viable and fertile at least in the heterozygous form (we did not confirm homozygosity due to budget constraints).
d. A survival assay (C. elegans individuals kept at 20° C. and fed with E. coli OP50) was conducted to assess resistance to senescence (if any) in the hil-1 mutant strain with respect to the wild-type C. elegans (strain N2) used as a negative control for the CRISPR/Cas genome editing. The results obtained showed a significant increase in lifespan for the C. elegans mutant strain (χ²=4.58; corrected P-value=0.032) when compared to the C. elegans N2 strain (see TABLE 2).

TABLE 2

| days after hatching | % alive (wt N2) | % alive (mutant hil-1) |
|---|---|---|
| 0.0 | 100 | 100 |
| 1.0 | 100 | 100 |
| 1.9 | 100 | 100 |
| 3.1 | 100 | 100 |
| 4.2 | 100 | 100 |
| 6.0 | 96.7 | 100 |
| 8.1 | 88.3 | 100 |
| 10.2 | 81.7 | 100 |
| 12.0 | 71.7 | 100 |
| 14.0 | 50.0 | 100 |
| 16.1 | 13.3 | 100 |
| 18.2 | 3.3 | 100 |
| 20.0 | 1.7 | 98.3 |
| 22.1 | 1.7 | 98.3 |
| 24.2 | 0.0 | 98.3 |

For a person skilled in the art it would be obvious that, given the well-known signs of senescence in C. elegans observable shortly after the individual reaches its adult form, the increased lifespan in the hil-1 mutant strain with respect to the wild-type C. elegans (strain N2) also implies the hil-1 mutant strain is significantly resistant to senescence, thereby demonstrating the industrial applicability of the present invention in vivo.

What is claimed is:

1. A method for producing an artificial protein sequence for histone H1 variants to induce resistance and/or protection against senescence, and/or age-related health conditions wherein the method comprises the steps of:
a. selecting a wild-type histone H1.0 or H1x protein sequence, or the wild-type sequence of a respective protein ortholog in the species of interest;
b. within the sequence selected in step a, recognizing the subsequences determined by regions or individual sites in the globular domain of the protein that conform the DNA-binding site of the histone H1.0 or H1x proteins, particularly the amino acid residues directly or indirectly interacting with the DNA;
c. applying a set of at least one amino acid substitutions, insertions, and/or deletions to one or more of the amino acid subsequences corresponding to the regions or sites recognized in step b, where the modifications do not alter the structure of the α-helical motifs and where the respective net electric charge (z) associated to each resulting modified amino acid subsequence is greater than before the modifications; and
d. obtaining the artificial protein sequence by applying the set of at least one amino acid substitutions, insertions, and/or deletions determined by step c into the wild-type histone H1.0, histone H1x, or respective orthologous protein sequence selected in step a, thereby producing the complete artificial protein sequence.

2. The method according to claim 1, wherein the increase of net electric charge (z) in step c is estimated particularly at physiological pH.

3. The method according to claim 2, wherein an artificial nucleic acid sequence that encodes the artificial protein sequence obtained in step d is produced.

4. The method according to claim 1, wherein depending on the variant of the wild-type histone H1.0 or H1x protein sequence selected in step a, it is recognized:
i. the first α-helical motif α₁ by using as a sequence homology guide the amino acid sequence SEQ. ID No. 1 if the wild-type histone variant is H1.0 or the amino acid sequence SEQ. ID No. 4 if the wild-type histone variant is H1x;
ii. the second α-helical motif α₂ by using as a sequence homology guide the amino acid sequence SEQ. ID No. 2 if the wild-type histone variant is H1.0 or the amino acid sequence SEQ. ID No. 5 if the wild-type histone variant is H1x;
iii. the third α-helical motif α₃ by using as a sequence homology guide the amino acid sequence SEQ. ID No. 3 if the wild-type histone variant is H1.0 or the amino acid sequence SEQ. ID No. 6 if the wild-type histone variant is H1x.

5. The method according to claim 4, wherein within each α-helical motif identified in steps i, ii, and iii, a set of at least one amino acid substitution sites is defined as follows: (S1,α₃,12), (S2,α₃,13), (S3,α₂,1), (S4,α₁,1), (S5,α₃,1), (S6,α₂,3), (S7,α₃,3), (S8,α₃,5), (S9,α₃,9), (S10,α₂,2) and (S11,α₃,11); where each triplet shows the substitution site, the α-helical motif, and its relative position (counting from N- to C-terminus) within the α-helical motif.

6. The method according to claim 5, wherein the amino acid substitution sites S1, S2, S3, S4, S5, S6, S7, S8, S9, S10, and S11 are mapped into the wild-type protein sequence selected in step a with respect to its three α-helix subsequences α₁, α₂, and α₃ using SEQ. ID No. 1-6.

7. The method according to claim 6, wherein the amino acid substitutions are optimized by using alternative substitute residues with the same rationale of increased net electric charge (z), particularly at physiological pH, in the artificial-sequence histone H1.0/H1x while preserving the secondary structure and overall function of the wild-type histone H1.0/H1x.

8. The method according to claim 7, wherein once mapping the substitution sites, a set of at least one to up to eleven amino acid substitutions is applied into the wild type protein sequence according to the following criteria: S1((K,S,T);R), S2((S,T,M,L);R), S3((K,L);R); S3((S,T);P); S4((S,T);P); S5(K;M); S5((S,T);N); S6((S,T);A); S7((D,E);N); S8((S,T,Y);R); S9((S,T);A) S10(Y;R); and S11(¬R;R), where for each substitution site, in the first part of the duplex it is shown the possible amino acid residues that can be found in the wild type sequences, and the second part it is shown the preferred substitute amino acid, and where ¬R denotes an amino acid residue other than R.

9. The method according to claim 8, wherein it is verified that the set of amino acid substitutions applied satisfies the condition of increased net electric charge (z), particularly at physiological pH, by estimating z for each modified α-helical motif at physiological pH and comparing it to the z estimate at physiological pH for its wild-type counterpart when the artificial-sequence α-helical motif and the wild-type α-helical motif are each in their respective post-translationally unmodified forms or when each is subjected to plausible PTMs.

10. The method according to claim 8, wherein the amino acid substitutions, insertions, and/or deletions are intended to redesign of the histone H1 α-helical motifs α₃ (most preferred, which binds to both nucleosomal and linker DNA), α₂ (second most preferred, which binds to nucleosomal DNA), and α₃ (third most preferred, which binds to linker DNA), and in particular to stabilize or enhance the electrostatic binding affinity of the α-helical motifs to nucleosomal and/or linker DNA.

11. An artificial histone H1.0 or H1x protein sequence for inducing resistance and/or protection against senescence, and/or age-related health conditions wherein the artificial protein sequence contains a set of at least one amino acid substitutions, insertions, and/or deletions to the DNA-binding site of the histone H1.0 or H1x proteins in the α-helical regions, where the substitutions, insertions, and/or deletions do not alter the structure of the α-helices and entail an increase in the net electric charge (z), particularly at physiological pH, of the resulting artificial-sequence protein.

12. An artificial protein sequence according to claim 11 wherein the increase in net electric charge (z) is estimated particularly at physiological pH.

13. An artificial protein sequence according to claim 11 wherein the DNA binding sites are located in the first, second, and/or third (counting from N- to C-terminus) α-helices of the histone H1.0 and histone H1x proteins.

14. An artificial protein sequence according to claim 13 wherein:
the amino acid sequence that corresponds to the first α-helix, denoted by α₁, of the wild-type histone H1 protein counterpart is identical or homologous to SEQ. ID. No. 1 if the wild-type histone variant is H1.0 or to the SEQ. ID. No. 4 if the wild-type histone variant is H1x;
the amino acid sequence that corresponds to the second α-helix, denoted by α₂, of the wild-type histone H1 protein counterpart is identical or homologous to SEQ. ID. No. 2 if the wild-type histone variant is H1.0 or to SEQ. ID. No. 5 if the wild-type histone variant is H1x; and
the amino acid sequence that corresponds to the third α-helix, denoted by α₃, of the wild-type histone H1 protein counterpart is identical or homologous to SEQ. ID. No. 3 if the wild-type histone variant is H1.0 or to SEQ. ID. No. 6 if the wild-type histone variant is H1x.

15. An artificial protein sequence according to claim 14, wherein the set of amino acid modification corresponds to at least one to up to eleven amino acid substitutions within the binding site in the α-helical motif.

16. An artificial protein sequence according to claim 15, wherein the eleven amino acid substitution sites S1 to S11 comprise at least one substitution for each of the first, second, and third α-helical motifs selected from: (S1,α₃,12), (S2,α₃,13), (S3,α₂,1), (S4,α₁,1), (S5,α₃,1), (S6,α₂,3), (S7,α₃,3), (S8,α₃,5), (S9,α₃,9), (S10,α₂,2) and (S11,α₃,11); where each triplet shows the substitution site, the α-helical motif and their relative position (counting from N- to C-terminus).

17. An artificial protein sequence according to claim 16, wherein the substitute amino acid residue are selected from alanine, methionine, leucine and arginine, for any substitution site or proline for substitution sites S3 and/or S4.

18. A synthetic or recombinant nucleic acid sequence including the cDNA and RNA codifying such sequences which encodes an artificial protein or an artificial peptide sequence according to any of the claims 11 to 17.

19. Use of the artificial protein sequence according to any of the claims 11 to 17 for analyzing and/or diagnosing senescence, and/or age-related health conditions in multicellular species such as the human species, other animal species, or plant species.

20. Use of the artificial protein sequence according to any of the claims 11 to 17 for inducing resistance and/or protection against senescence, and/or age-related health conditions in multicellular species such as the human species, other animal species, or plant species.

21. Use according to claim 20 wherein the resistance and/or protection includes but is not limited to the arrest, slowdown, and/or prevention of senescence, and/or age-related health conditions in multicellular species such as the human species, other animal species, or plant species.

22. Use of the artificial protein sequence according to claim 21, wherein the age-related health conditions are selected from age-related cancer, atherosclerosis and cardiovascular disease, arthritis, cataracts, osteoporosis, type-2 diabetes, hypertension, Alzheimer's disease, benign prostate hyperplasia, hearing disability, age-related macular degeneration, neurodegenerative diseases, degenerative diseases, immune senescence diseases, skin aging, and skin wrinkles.

23. Use of the artificial protein sequence according to any of the claims 11 to 17 for biomedical, cosmetic, industrial, and/or agricultural applications.


SEQUENCE LISTING (25 sequences)

SEQ ID NO: 1 - protein, 13 aa, H. sapiens
KYSDMIVAAIQAE

SEQ ID NO: 2 - protein, 12 aa, H. sapiens
RQSIQKYIKSHY

SEQ ID NO: 3 - protein, 16 aa, H. sapiens
NADSQIKLSIKRLVTT

SEQ ID NO: 4 - protein, 13 aa, H. sapiens
KYSQLVVETIRRL

SEQ ID NO: 5 - protein, 12 aa, H. sapiens
LAKIYTEAKKVP

SEQ ID NO: 6 - protein, 16 aa, H. sapiens
NGRTYLKYSIKALVQN

SEQ ID NO: 7 - protein, 232 aa, Caenorhabditis elegans
MTTSLIHMANHLDASTEEISLNYVLLGHPHHERAQHHPSYMDMIKGAIQAIDNGTGSSKAAILKYIAQNYHVGENLPKVNNHLRSVLKKAVDSGDIEQTRGHGATGSFRMGKECEKNLQVGIPVQTKPMLMLKEVRQKLENISKAEKTKPSTSSMSTNKKGKPISTMKKRGVMSKKRSSKNKMAPKAKSHGLKKKGPATKSSGLVHKAAGAKNEAAPTTKMELRTGTRKSYC

SEQ ID NO: 8 - protein, 232 aa, C. elegans
MTTSLIHMANHLDASTEEISLNYVLLGHPHHERAQHHPPYMDMIKGAIQAIDNGTGSSRAAILKYIAQNYHVGENLPKVNNHLRSVLKRAVDSGDIEQTRGHGATGSFRMGKECEKNLQVGIPVQTKPMLMLKEVRQKLENISKAEKTKPSTSSMSTNKKGKPISTMKKRGVMSKKRSSKNKMAPKAKSHGLKKKGPATKSSGLVHKAAGAKNEAAPTTKMELRTGTRKSYC

SEQ ID NO: 9 - protein, 194 aa, Mus musculus
MTENSTSAPAAKPKRAKASKKSTDHPKYSDMIVAAIQAEKNRAGSSRQSIQKYIKSHYKVGENADSQIKLSIKRLVTTGVLKQTKGVGASGSFRLAKGDEPKRSVAFKKTKKEVKKVATPKKAAKPKKAASKAPSKKPKATPVKKAKKKPAATPKKAKKPKVVKVKPVKASKPKKAKTVKPKAKSSAKRASKKK

SEQ ID NO: 10 - protein, 194 aa, M. musculus
MTENSTSAPAAKPKRAKASKKSTDHPKYSDMIVAAIQAEKNRAGSSRQSIQKYIKSHYKVGENADSQIKLSIKRRVTTGVLKQTKGVGASGSFRLAKGDEPKRSVAFKKTKKEVKKVATPKKAAKPKKAASKAPSKKPKATPVKKAKKKPAATPKKAKKPKVVKVKPVKASKPKKAKTVKPKAKSSAKRASKKK

SEQ ID NO: 11 - protein, 194 aa, M. musculus
MTENSTSAPAAKPKRAKASKKSTDHPKYSDMIVAAIQAEKNRAGSSRQAIQKYIKSHYKVGENANSQIKLSIKRRVTTGVLKQTKGVGASGSFRLAKGDEPKRSVAFKKTKKEVKKVATPKKAAKPKKAASKAPSKKPKATPVKKAKKKPAATPKKAKKPKVVKVKPVKASKPKKAKTVKPKAKSSAKRASKKK

SEQ ID NO: 12 - protein, 194 aa, Homo sapiens
MTENSTSAPAAKPKRAKASKKSTDHPKYSDMIVAAIQAEKNRAGSSRQSIQKYIKSHYKVGENADSQIKLSIKRLVTTGVLKQTKGVGASGSFRLAKSDEPKKSVAFKKTKKEIKKVATPKKASKPKKAASKAPTKKPKATPVKKAKKKLAATPKKAKKPKTVKAKPVKASKPKKAKPVKPKAKSSAKRAGKKK

SEQ ID NO: 13 - protein, 194 aa, H. sapiens
MTENSTSAPAAKPKRAKASKKSTDHPKYSDMIVAAIQAEKNRAGSSRQSIQKYIKSHYKVGENADSQIKLSIKRRVTTGVLKQTKGVGASGSFRLAKSDEPKKSVAFKKTKKEIKKVATPKKASKPKKAASKAPTKKPKATPVKKAKKKLAATPKKAKKPKTVKAKPVKASKPKKAKPVKPKAKSSAKRAGKKK

SEQ ID NO: 14 - protein, 213 aa, H. sapiens
MSVELEEALPVTTAEGMAKKVTKAGGSAALSPSKKRKNSKKKNQPGKYSQLVVETIRRLGERNGSSLAKIYTEAKKVPWFDQQNGRTYLKYSIKALVQNDTLLQVKGTGANGSFKLNRKKLEGGGERRGAPAAATAPAPTAHKAKKAAPGAAGSRRADKKPARGQKPEQRSHKKGAGAKKDKGGKAKKTAAAGGKKVKKAAKPSVPKVPKGRK

SEQ ID NO: 15 - protein, 213 aa, H. sapiens
MSVELEEALPVTTAEGMAKKVTKAGGSAALSPSKKRKNSKKKNQPGKYSQLVVETIRRLGERNGSSLAKIYTEAKKVPWFDQQNGRTYLKYSIKRRVQNDTLLQVKGTGANGSFKLNRKKLEGGGERRGAPAAATAPAPTAHKAKKAAPGAAGSRRADKKPARGQKPEQRSHKKGAGAKKDKGGKAKKTAAAGGKKVKKAAKPSVPKVPKGRK

SEQ ID NO: 16 - protein, 194 aa, Rattus norvegicus
MTENSTSTPAAKPKRAKAAKKSTDHPKYSDMIVAAIQAEKNRAGSSRQSIQKYIKSHYKVGENADSQIKLSIKRLVTTGVLKQTKGVGASGSFRLAKGDEPKRSVAFKKTKKEVKKVATPKKAAKPKKAASKAPSKKPKATPVKKAKKKPAATPKKAKKPKIVKVKPVKASKPKKAKPVKPKAKSSAKRASKKK

SEQ ID NO: 17 - protein, 194 aa, R. norvegicus
MTENSTSTPAAKPKRAKAAKKSTDHPKYSDMIVAAIQAEKNRAGSSRQSIQKYIKSHYKVGENADSQIKLSIKRRVTTGVLKQTKGVGASGSFRLAKGDEPKRSVAFKKTKKEVKKVATPKKAAKPKKAASKAPSKKPKATPVKKAKKKPAATPKKAKKPKIVKVKPVKASKPKKAKPVKPKAKSSAKRASKKK

SEQ ID NO: 18 - protein, 192 aa, Rattus norvegicus
MSVELEEALPPTSADGTARKTAKASGSAAPTQPKRRKNRKKNQPGKYSQLVVETIRKLGERGGSSLARIYAEARKVAWFDQQNGRTYLKYSIRALVQNDTLLQVKGTGANGSFKLNRKKLEGSAEKRGASAASSPAPKARTAAAAAADRTPARPQPERRAQKSKKAAAAAASTKKVKKAAKPSVPKVPKGRK

SEQ ID NO: 19 - protein, 192 aa, R. norvegicus
MSVELEEALPPTSADGTARKTAKASGSAAPTQPKRRKNRKKNQPGKYSQLVVETIRKLGERGGSSLARIYAEARKVAWFDQQNGRTYLKYSIRRRVQNDTLLQVKGTGANGSFKLNRKKLEGSAEKRGASAASSPAPKARTAAAAAADRTPARPQPERRAQKSKKAAAAAASTKKVKKAAKPSVPKVPKGRK

SEQ ID NO: 20 - protein, 194 aa, Nothobranchius furzeri
MAETSGAPAKAKKASKPKKPASHPKYSDMIKAAIVHDASRSGASRQSIQKYVRKTYKVGDNADVQIKMALKRLVASGMLRHTKGIGASGSFRLTKPEDSKKPAKAAASAKPKKVAKPKPKKAAKPKKVPKTPEKPKKAAAKKVKKVAKKATPAKAKKAPAKKSKAAKPKAKPAKKAAKPKAKTPKKAAKSAKKK

SEQ ID NO: 21 - protein, 194 aa, N. furzeri
MAETSGAPAKAKKASKPKKPASHPKYSDMIKAAIVHDASRSGASRQAIQKYVRKTYKVGDNANVQIKMALKRRVASGMLRHTKGIGASGSFRLTKPEDSKKPAKAAASAKPKKVAKPKPKKAAKPKKVPKTPEKPKKAAAKKVKKVAKKATPAKAKKAPAKKSKAAKPKAKPAKKAAKPKAKTPKKAAKSAKKK

SEQ ID NO: 22 - protein, 256 aa, Drosophila melanogaster
MSDSAVATSASPVAAPPATVEKKVVQKKASGSAGTKAKKASATPSHPPTQQMVDASIKNLKERGGSSLLAIKKYITATYKCDAQKLAPFIKKYLKSAVVNGKLIQTKGKGASGSFKLSASAKKEKDPKAKSKVLSAEKKVQSKKVASKKIGVSSKKTAVGAADKKPKAKKAVATKKTAENKKTEKAKAKDAKKTGIIKSKPAATKAKVTAAKPKAVVAKASKAKPAVSAKPKKTVKKASVSATAKKPKAKTTAAKK

SEQ ID NO: 23 - protein, 256 aa, D. melanogaster
MSDSAVATSASPVAAPPATVEKKVVQKKASGSAGTKAKKASATPSHPPTQQMVDASIKNLKERGGSSRLAIKKYITATYKCDAQMLAPFIKKYLKRAVVNGKLIQTKGKGASGSFKLSASAKKEKDPKAKSKVLSAEKKVQSKKVASKKIGVSSKKTAVGAADKKPKAKKAVATKKTAENKKTEKAKAKDAKKTGIIKSKPAATKAKVTAAKPKAVVAKASKAKPAVSAKPKKTVKKASVSATAKKPKAKTTAAKK

SEQ ID NO: 24 - protein, 167 aa, Arabidopsis thaliana
MAEDKILKKTPAAKKPRKPKTTTHPPYFQMIKEALMVLKEKNGSSPYAIAKKIEEKHKSLLPESFRKTLSLQLKNSVAKGKLVKIRASYKLSDTTKMITRQQDKKNKKNMKQEDKEITKRTRSSSTRPKKTVSVNKQEKKRKVKKARQPKSIKSSVGKKKAMKASAA

SEQ ID NO: 25 - protein, 167 aa, A. thaliana
MAEDKILKKTPAAKKPRKPKTTTHPPYFQMIKEALMVLKEKNGSSPRAIAKKIEEKHKSLLPENFRKRLSLQLKNRVAKGKLVKIRASYKLSDTTKMITRQQDKKNKKNMKQEDKEITKRTRSSSTRPKKTVSVNKQEKKRKVKKARQPKSIKSSVGKKKAMKASAA